(19) United States
(12) Patent Application Publication
(10) Pub. No.: US 2012/0301482 A1
(43) Pub. Date: Nov. 29, 2012

(54) METHODS AND COMPOSITIONS FOR TREATMENT OF LUNG INJURY

(75) Inventor: Stijn P. Delanghe, Denver, CO (US)

(73) Assignee: NATIONAL JEWISH HEALTH, Denver, CO (US)

(21) Appl. No.: 13/392,134

(22) PCT Filed: Aug. 25, 2010
(86) PCT No.: PCT/US10/46607

§ 371 Date: Mar. 29, 2012

Related U.S. Application Data
(60) Provisional application No. 61/236,676, filed on Aug. 25, 2009.

(51) Int. Cl. A61K 38/18; A61K 35/42; G01N 33/566; A61P 11/06; A61P 29/00; A61K 39/395; A61P 11/00

(52) U.S. Cl. 424/172.1; 424/93.7; 514/9.1; 435/7.21

(57) ABSTRACT

The present invention provides a method to treat a condition related to injury of lung epithelial cells in a subject by administering a compound that binds FgfR2b, for example to promote proliferation of lung epithelial stem cells, as well as such compounds in pharmaceutical formulations. The invention also provides for methods to treat a condition related to injury of lung epithelial cells by isolating cells expressing FgfR2b, multiplying the cells and introducing the multiplied cells for repair of lung injury. Related methods for detecting lung injury by detecting the level of expression of FgfR2b well as for treating a condition related to proliferation of lung epithelial cells by administering a compound that binds to FgfR2b, to block receptor signaling are also provided.

### CROSS REFERENCE TO RELATED APPLICATION

The present application claims the benefit of priority under 35 U.S.C. §119(e) from Provisional Patent Application Ser. No. 61/236,676, filed on Aug. 25, 2009. Provisional Patent Application Ser. No. 61/236,676 is incorporated herein by reference in its entirety.

### GOVERNMENT SUPPORT

This invention was supported in part with funding provided by NIH Grant Nos. 1R01HL092967-01A1, 1R01HL092967-01A1S1 and 1R01HL074832-05A1 all awarded by the National Institutes of Health. The government may have certain rights to this invention.

### FIELD OF THE INVENTION

The field of the present invention is related to epithelial regeneration in response to lung injury, specifically in the proliferation of lung epithelial stem cells (LESCs) during epithelial regeneration.

### BACKGROUND OF THE INVENTION

Remodeling of the airway epithelium is a common pathological feature in chronic lung disease and a predisposing factor in the development of lung cancer. Accordingly, understanding cellular and molecular mechanisms of epithelial maintenance and repair are fundamental to the development of therapeutic modalities for the treatment of chronic lung disease.

Throughout adult life, multi-cellular organisms must generate new cells to maintain the structural and functional integrity of their tissues. In young animals, tissue damage can usually be repaired quickly, but this natural capacity may fail after repeated challenges and with age. Diseases such as cancer may exploit the mechanisms by which the body normally rebuilds itself.

The lung has a complex three-dimensional structure that features major differences along its proximodistal axis in terms of the composition of the endoderm-derived epithelium. The trachea and primary lung buds arise by different morphogenetic processes from contiguous regions of the embryonic foregut (Cardoso, W. V., and Lu, J. (2006) Regulation of early lung morphogenesis: questions, facts and controversies. Development 133, 1611-1624). In the adult mouse trachea and primary bronchi (cartilaginous airways), the luminal epithelium contains two main columnar epithelial cell types: ciliated cells and Clara cells. The Clara cells produce secretoglobins, the most abundant of which is Scgb1a1 (also known as CCSP, SCGB1A1 or CCA). A distinguishing feature of the cartilaginous airways is that they contain a discontinuous population of relatively unspecialized basal cells that express p63 and specific keratins (K14 and K5). The more distal airways (small bronchi and bronchioles) have a columnar epithelium in which Clara cells predominate over ciliated cells and where there are more neurendocrine (NE) cells than in the trachea. Importantly however, these airways lack basal cells, therefore excluding a potential role for these cells in local turnover and repair (Pack, R. J., Al-Ugaily, L. H., and Morris, G. (1981) The cells of the tracheobronchial epithelium of the mouse: a quantitative light and electron microscope study. J Anat 132, 71-84).

The adult lung is a vital and complex organ that normally turns over very slowly. The epithelial cells that line the airways are constantly exposed to potential toxic agents and pathogens in the environment, and they must therefore be able to respond quickly and effectively to both cellular damage and to local production of immune cytokines. The cellular hallmark of lung repair after injury of lung epithelial cells, such as by naphthalene, is a rapid proliferative response ultimately leading to restoration of the airway epithelium and function. The origin of the cells that replace the injured airway epithelium have been shown to be naphthalene resistant or variant Clara cells (Clara^{V}) located at the bronchio-alveolar duct junctions (BADJs) and neuroendocrine bodies (NEBs). However, little is known about the activation mechanism of these latent “stem cells” (reviewed in: Chen, H., Matsumoto, K., and Stripp, B. R. (2009) Bronchiolar progenitor cells. Proc Am Thorac Soc 6, 602-606; and Rawlins, E. L., and Hogan, B. L. (2006) Epithelial stem cells of the lung: privileged few or opportunities for many? Development 133, 2455-2465).

A popular and clean lung injury model is the destruction of Clara cells that express cytochrome P4502F2 (Cyp2f2) by naphthalene. Although there are some strain and dose variations in the response, the sequence of events is approximately as follows. Within a few hours all of the Clara cells die, leaving intact the few Clara cells that do not express Cyp2f2 and are therefore resistant. Cell proliferation begins 2-3 days after injury and by 2-4 weeks the epithelium returns to a steady state. Different mechanisms appear to operate for the renewal of the Clara cells depending on the region of the lung in which the repair occurs. In the trachea and main bronchi, there is good evidence that basal cells function as stem cells in repairing the destruction of Clara cells by naphthalene. In the more distal lung, where there are no basal cells, there is evidence that the Clara cell population is restored by proliferation and self renewal of a small number of resistant or Clare cells that survive naphthalene injury. The precise mechanisms that underlie the self renewal of Clare cells and the lineage diversification of their progeny are currently unknown.

Fibroblast growth factors (Fgfs) are a family of 22 growth factors that possess broad mitogenic and cell survival activities, and are involved in a variety of biological processes, including embryonic development, cell growth, morphogenesis, tissue repair, tumor growth and invasion. The Fgfs exert their diverse actions by binding, dimerizing, and activating members of the Fgf receptor (FgfRs) family of receptor tyrosine kinases. Both Fgfs and FgfRs interact with heparin sulfate glycosaminoglycans for sustainable Fgf-FgfR binding and dimerization to occur. The Fgfs differ in size and sequence but all contain a core region of homology encompassing 120-130 residues. Based on sequence comparison, the 22 known mammalian Fgfs (Fgf1-Fgf14 and Fgf16-Fgf23) are grouped into eight subfamilies. (Olsen S. K., Garbi, M., Zampieri N., Eliseenkova, A. V., Ornitz, D. M., Goldfarb, M., and Mohammadi, M. (2003) Fibroblast Growth Factor (FGF) Homolgous Factors Share Structural but Not Functional Homology with FGFs JBC 278, 34226-34236).

Fibroblast growth factor 10, also known as Fgf10, is a member of the Fgf7 subfamily which includes Fgf3, Fgf7, Fgf10 and Fgf22. Fgf10 is also thought to be required for embryonic epidermal morphogenesis including brain development, lung morphogenesis, and initiation of limb bud formation and is also implicated to be a primary factor in the process of wound healing. During lung development Fgf10 is secreted by the parabronchial smooth muscle cell (PSMC) progenitors in the distal mesenchyme. Fgf10 binds fibroblast growth factor receptor 2b (FgfR2b) present on the distally located epithelial progenitor cells and activates the canonical WNT signaling pathway in the cells. This signaling is essential for the maintenance and proliferation of the epithelial progenitor cells. Additionally, β-catenin signaling in the PSMC progenitors is also essential for the maintenance and proliferation of the epithelial progenitor cells. In addition, PSMCs in the lung are derived from Fgf/expressing cells in the distal mesenchyme during early lung development (Mailleux, A. A., Kelly, R., Veltmaat, J. M., De Langhe, S. P., Zaffran, S., Thiery, J. P., and Bellusci, S. (2005) Fgf10 expression identifies parabronchial smooth muscle cell progenitors and is required for their entry into the smooth muscle cell lineage. Development 132, 2157).

In addition, amplification of PSMC progenitors as well as their Fgf10 expression is regulated by mesenchymal β-catenin signaling and deletion of β-catenin leads to their premature differentiation into PSMCs (De Langhe, S. P., Carraro, G., Tefft, D., Li, C., Xu, X., Chai, Y., Minoo, P., Hajihosseini, M. K., Drouin, J., Kaartinen, V., et al. (2008) Formation and Differentiation of Multiple Mesenchymal Lineages during Lung Development Is Regulated by beta-catenin Signaling. PLoS ONE 3, e1516.

### SUMMARY OF THE INVENTION

The present invention provides for a method to treat a condition related to injury of lung epithelial cells in a subject comprising a compound that binds FGfR2b.

In another aspect of the present invention the compound is administered by inhalation.

The present invention also provides for a method to treat a condition related to injury of lung epithelial cells in a subject. This method comprises isolating LESCs expressing FGfR2b from a donor. The method further comprises culturing the LESCs expressing FgfR2b to multiply them. This method further comprises introducing the cultured LESCs expressing FgfR2b into the subject. In another aspect of this method the step of culturing the LESCs expressing FgfR2b to multiply them further comprises exposure of the LESCs to a compound that binds FgfR2b. In another aspect of this method, the LESCs are isolated by contacting with an antibody specific for FgfR2b. This method further comprises a donor that is the subject.

In another aspect of the methods of the present invention the compound is an agonist of FgfR2b. The methods of the present invention also provide that the compound is an antibody. The methods of the present invention can also provide that the compound is a fibroblast growth factor (Fgf). The methods of the present invention can also provide that the compound can be Fgf1, Fgf3, Fgf7, Fgf9, Fgf10, and Fgf22. The methods of the present invention can also provide that the compound is Fgf10. The methods of the present invention can also provide that the compound is a fragment of Fgf capable of binding FgfR2b.

In another aspect of the methods of the present invention the compound binding FgfR2b stimulates proliferation of LESCs expressing FgfR2b.

The present invention also provides for a method to detect lung injury in a subject. This method comprises detecting the level of expression of FgfR2b in a subject sample wherein an elevated level of expression of FgfR2b is indicative of lung injury.

In another embodiment of the methods of the present invention the condition related to injury of lung epithelial cells can be asthma, inflammation of the lungs, a condition associated with exposure to environmental toxins, a condition associated with exposure to bacteria, a condition associated with exposure to a virus, cystic fibrosis, a pneumonectomy and bleomycin mediated epithelial injury.

In yet another embodiment of the methods of the present invention the condition related to injury of lung epithelial cells in a subject is a condition associated with exposure to environmental toxins wherein the environmental toxin can be naphthalene, ozone, smoke, tobacco smoke, chemical fumes, exhaust, mustard gas, acid, aromatic hydrocarbons and radiation.

The present invention also provides for a pharmaceutical composition comprising a compound that binds FgfR2b and a pharmaceutically acceptable carrier. This method further provides that the compound is an agonist of FgfR2b. This method further provides that the compound is a fibroblast growth factor (Fgf). The method further provides that the compound is Fgf1, Fgf3, Fgf7, Fgf9, Fgf10, Fgf22 or a fragment of an Fgf capable of binding FgfR2b. This method further provides that the agonist of FgfR2b is an antibody.

The present invention also provides for a method to treat a condition related to proliferation of lung epithelial cells in a subject, by administering a compound that binds to FgfR2b to block receptor signaling. This method further provides that the blocking of the receptor signaling occurs by preventing dimerization of FgfR2b. This method further provides that the compound is an antibody. This method further provides that the compound is a cytotoxic agent.

In yet another aspect the methods of the present invention the subject is human.

### BRIEF DESCRIPTION OF THE DRAWINGS

FIG. 1 shows Real-Time quantitative PCR (qPCR) analysis of Wnt7b mRNA abundance in 2 month old wild-type lungs 3 days after corn oil treatment (3d corn oil) compared to naphthalene treatment (3d npt). RNA was isolated from mice lung accessory lobes.

FIG. 2 shows qPCR analysis of Scgb1a1 mRNA abundance of adult lungs from control (ctrl), Rosa26-rtTA; Tet-sFgfr2b (Tet-sFgfr2b), Rosa26rtTA; Tet-Fgf10 (Tet-Fgf10), Rosa26rtTA;Tet-Dkk1 (Tet-Dkk1) and Rosa26-rtTA;Tet-Dkk1; Tet-Fgf10 (Tet-Dkk1+Tet-Fgf10) mice, 3 days, 7 days and 14 days after naphthalene treatment.

FIG. 3 shows qPCR analysis of Scgb1a1 mRNA abundance in 2 month old smMHC-Cre⁻;Fgf10^{f/f} and smMHC-Cre;Fgf10^{f/f} mice 14 days after naphthalene treatment.

FIG. 4 shows phosph-β-cateninSer55 mean gray values for immunostaining on lungs from Rosa26-rtTA (CTRL), Rose26-rtTA; Tet-Fgf10 (Fgf10), and Rosa26rtTA; Tet-sFgfr2b(Fgfr2b) mice, 14 days after naphthalene treatment.

### DESCRIPTION OF INVENTION

The present inventors have made the novel discovery that after epithelial lung injury, mature PSMCs show activated β-catenin signaling, start to proliferate and re-express Fgf10, thus recapitulating an embryonic PSMC progenitor like phenotype. The Fgf10 secreted from the PSMCs in turn further activates LESCs at the bronchio-alveolar duct junctions (BADJs) in a paracrine fashion and promotes epithelial regeneration in damaged airway epithelium.

As described herein, upon lung injury, the PSMCs underwent massive proliferation as evidenced by BrdU incorporation. The PSMCs also showed activation of the Wnt signaling and β-catenin signaling pathway as evidenced by increased TOPGAL activity, and expressed Fgf10. The activation of β-catenin activity in the PSMCs, as well as their massive proliferation reported herein, are two very uncharacteristic features of mature differentiated PSMCs. It appears that in response to injury, the mature PSMCs recapitulated an Fgf10 expressing embryogenic PSMC progenitor like phenotype.

Furthermore, Fgf10 expressed by the PSMCs stimulated the proliferation of the epithelial LESCs in a paracrine fashion by binding to FgfR2b. Inhibition of Fgf10 signaling by over-expression of a soluble FgfR2b receptor, which would inhibit the Fgf10 binding to the FgfR2b cell surface receptor and disrupt the Fgf10 signaling, negatively affected epithelial regeneration after injury. Additionally, overexpression of Fgf10 improved epithelial repair. As described in the examples below, in injured lungs in which Fgf10 was overexpressed, the number of LESCs present near the BADJ was markedly increased when compared with naphthalene injured lungs in which Fgf10 expression was not induced. Fgf10 expression by the PSMCs also induced the expression of embryonic epithelial progenitor markers in the LESCs.

To summarize, the present inventors have shown herein that upon injury of lung epithelial cells, Fgf10 secreted from PSMCs activates the Fgf10 mediated signaling pathway in LESCs by binding to Fgf10R2b, and promotes proliferation of LESCs which results in the re-epithalization of the damaged airway epithelium.

The present invention relates to a method to treat a condition related to injury of lung epithelial cells in a subject by administering a compound that binds FgfR2b. As used herein, “lung injury” or “injury of lung epithelial cells” refers to cell death of lung cells and particularly, lung epithelial cells. Conditions related to injury of lung epithelial cells for which methods of the present invention are useful include any condition which causes or results in lung injury. Examples of such conditions include but are not limited to asthma, inflammation of the lungs, conditions associated with exposure to environmental toxins, conditions associated with exposure to bacteria, conditions associated with exposure to a virus, cystic fibrosis, a pneumonectomy and bleomycin mediated epithelial injury. Examples of environmental toxins include but are not limited to naphthalene, ozone, smoke, tobacco smoke, chemical fumes, exhaust, mustard gas, acid, aromatic hydrocarbons and radiation.

As used herein the phrase “to treat a condition” refers to clinical intervention in an attempt to alter the natural course of the subject or cell being treated, and may be performed either for prophylaxis and/or during the course of clinical pathology. Desirable effects include preventing occurrence or recurrence of disease or condition, alleviation of symptoms, diminishment of any direct or indirect pathological consequences of the disease or condition, preventing metastasis, lowering the rate of disease progression, amelioration or palliation of the disease state, and remission or improved prognosis. Accordingly, a therapeutic benefit is not necessarily a cure for a particular disease or condition, but rather, preferably encompasses a result which most typically includes alleviation of the disease or condition, elimination of the disease or condition, reduction of a symptom associated with the disease or condition, prevention or alleviation of a secondary disease or condition resulting from the occurrence of a primary disease, and/or prevention of the disease or condition.

A beneficial effect can easily be assessed by one of ordinary skill in the art and/or by a trained clinician who is treating the subject. The term “condition” refers to any deviation from the normal health of a mammal and includes a state when condition symptoms are present, as well as conditions in which a deviation (e.g. lung injury) has occurred, but symptoms are not yet manifested.

The method of the present invention can be used in any animal subject, and particularly, in any animal of the Vertebrate class, Mammalian, including, without limitation, humans, primates, rodents, livestock and domestic pets. Preferred mammals to treat using the methods of the present invention include humans.

Accordingly, the methods of the present invention include the use of a variety of compounds which increase the amount of proliferation of LESCs in the lungs of the mammal, particularly by activating FgfR2b in a cell such that proliferation of LESCs occurs. Compounds useful in the present invention include, for example, proteins, nucleic acid molecules, antibodies (including antigen-binding fragments), and small molecule pharmaceuticals.

Without intending to be bound by theory, the compound binding FgfR2b stimulates proliferation of the epithelial cell on which FgfR2b is a receptor, such as LESCs. LESCs are cells that have the potential to differentiate into lung epithelial cells. Examples of LESCs include but are not limited to bronchioalveolar stem cells (BASCs), basal cells and alveolar epithelial type II cells.

Proliferation of LESCs in a lung sample can be determined, for example, by a bromodeoxyuridine (BrdU) incorporation assay or by a phosphohistone H3 labeling assay. Alternatively, the proliferation of LESCs can be determined by determining the level of Scgb1a1 in a bronchio-alveoli lavage sample from the subject where an increase in Scgb1a1 correlates to proliferation of LESCs in the subject.

Without being bound by theory, FgfR2b biological activity can be identified using bioassays and molecular assays, including, but not limited to, phosphorylation assays, kinase assays, immunofluorescence microscopy, and combinations thereof.

In various embodiments of the present invention, methods include administering a compound that binds FgfR2b, i.e., an agonist of FgfR2b. According to the present invention, an FgfR2b agonist is any compound which increases or restores the biological activity of FgfR2b (i.e., as compared to the biological activity of FgfR2b prior to lung epithelial cell injury, preferably by direct binding to and activation of FgfR2b). Such a compound is effective to agonize the biological activity of FgfR2b, for example by binding to and activating FgfR2b on LESCs. The phrase “FgfR2b agonist” generally refers to any compound, including, but not limited to, an antibody that selectively binds to and activates or increases the activation of FgfR2b, as well as proteins, fragments, homologues, and small molecule pharmaceuticals, which is characterized by its ability to agonize (e.g., stimulate, induce, increase, enhance, activate) the biological activity of a naturally occurring FgfR2b (e.g., by interaction/binding with and/or activation of FgfR2b).

In preferred embodiments, FgfR2b agonists include but are not limited to antibodies specific to FgfR2b, an Fgf such as Fgf1, Fgf2, Fgf3, Fgf4, Fgf5, Fgf6, Fgf7, Fgf8, Fgf9, Fgf10, Fgf11, Fgf12, Fgf13, Fgf14, Fgf16, Fgf17, Fgf18, Fgf19, Fgf20, Fgf21, Fgf22 or Fgf23, fragments or homologs of an Fgf, or small molecule pharmaceuticals (also referred to as a “mimetic”). In preferred embodiments, when the compound is an Fgf, it is selected from Fgf1, Fgf3, Fgf7, Fgf9, Fgf10, or Fgf22 and particularly Fgf10.

According to the present invention, a fragment of an Fgf that is useful in the present invention is any fragment that binds to and still activates FgfR2b. For example, the native human Fgf10 peptide is 208 amino acids in length and therefore, one of skill in the art can readily produce and test fragments of Fgf10 that can serve as FgfR2b agonists.

Suitable homologues of the present invention can be identified in a straightforward manner by the ability of the homologue to bind FgfR2b, such as in any standard binding assay. An Fgf homologue can also be identified by its ability to activate FgfR2b.

Indeed, in one embodiment, the Fgf or other protein fragment or protein homologue that activates the FgfR2b can be provided as a nucleic acid molecule encoding the protein. According to the present invention, a nucleic acid molecule can include DNA, RNA, or derivatives of either DNA or RNA. A nucleic acid molecule of the present invention can include a ribozyme which specifically targets RNA encoding an Fgf. A nucleic acid molecule encoding a Fgf protein, fragment or homologue thereof can be obtained from its natural source, either as an entire (i.e., complete) gene or a portion thereof that is capable of encoding an Fgf protein, fragment or homologue thereof that increases the activity of FgfR2b thereby increasing proliferation of LESCs, when such protein and/or nucleic acid molecule encoding such protein is administered to the mammal. In one embodiment of the present invention, a nucleic acid molecule encoding an Fgf is an oligonucleotide that encodes a portion of an Fgf. Such an oligonucleotide can include all or a portion of a regulatory sequence of a nucleic acid molecule encoding an Fgf. A nucleic acid molecule can also be produced using recombinant DNA technology (e.g., polymerase chain reaction (PCR) amplification, cloning) or chemical synthesis. Nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications do not substantially interfere with the nucleic acid molecule's ability to encode an Fgf or a homologue thereof that is useful in the method of the present invention. An isolated, or biologically pure, nucleic acid molecule, is a nucleic acid molecule that has been removed from its natural milieu. As such, “isolated” and “biologically pure” do not necessarily reflect the extent to which the nucleic acid molecule has been purified.

A nucleic acid molecule homologue can be produced using a number of methods known to those skilled in the art (see, for example, Sambrook et al., Molecular Cloning: A Laboratory Manual, Cold Spring Harbor Labs Press, 1989). For example, nucleic acid molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid (e.g., Fgf activity, as appropriate).

Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding an Fgf, fragment or a homologue thereof. In addition, the phrase “recombinant molecule” primarily refers to a nucleic acid molecule operatively linked to a transcription control sequence, but can be used interchangeably with the phrase “nucleic acid molecule” which is administered to a mammal.

One compound useful in the method of the present invention includes an antibody or antigen binding fragment that selectively binds to FgfR2b. Such an antibody can selectively bind to any FgfR2b, including fragments of such receptors. According to the present invention, the phrase “selectively binds to” refers to the ability of an antibody, antigen binding fragment or binding partner of the present invention to preferentially bind to specified proteins (e.g. FgfR2b). More specifically, the phrase “selectively binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.

Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)2 fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention.

In general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). Modifications of a protein, such as in a homologue or fragment, may result in proteins having the same biological activity as the naturally occurring protein, or in proteins having decreased or increased biological activity as compared to the naturally occurring protein. Modifications which result in a decrease in protein expression or a decrease in the activity of the protein, can be referred to as inactivation (complete or partial), down-regulation, or decreased action of a protein. Similarly, modifications which result in an increase in protein expression or an increase in the activity of the protein, can be referred to as amplification, overproduction, activation, enhancement, up-regulation or increased action of a protein.

A small molecule pharmaceutical or mimetic refers to any peptide or non-peptide compound that is able to mimic the biological action of a naturally occurring peptide, often because the mimetic has a basic structure that mimics the basic structure of the naturally occurring peptide and/or has the salient biological properties of the naturally occurring peptide. Mimetics can include, but are not limited to: peptides that have substantial modifications from the prototype such as no side chain similarity with the naturally occurring peptide (such modifications, for example, may decrease its susceptibility to degradation); anti-idiotypic and/or catalytic antibodies, or fragments thereof; non-proteinaceous portions of an isolated protein (e.g., carbohydrate structures); or synthetic or natural organic molecules, including nucleic acids and drugs identified through combinatorial chemistry, for example. Such mimetics can be designed, selected and/or otherwise identified using a variety of methods known in the art. Various methods of drug design, useful to design mimetics or other therapeutic compounds useful in the present invention are disclosed in Maulik et al., 1997, Molecular Biotechnology: Therapeutic Applications and Strategies, Wiley-Liss, Inc.

In the methods of the present invention, the compound can be administered to a subject by any route of administration that is suitable for delivery of the compound. In accordance with the present invention, acceptable protocols to administer a compound including the route of administration and the effective amount of a compound to be administered to a mammal can be accomplished by those skilled in the art. A compound of the present invention can be administered in vivo or ex vivo. Suitable in vivo routes of administration can include, but are not limited to, oral, nasal, inhaled, topical, intratracheal, transdermal, rectal, and parenteral routes. Preferred parenteral routes can include, but are not limited to, subcutaneous, intradermal, intravenous, intramuscular, and intraperitoneal routes. Preferred topical routes include inhalation by aerosol (i.e., spraying) or topical surface administration to the skin of a mammal. Preferably, a compound is administered by nasal, inhaled (e.g., aerosol), intratracheal, oral, topical, or intraperitoneal routes. Ex vivo refers to performing part of the administration step outside of the subject, such as by contacting a population of cells removed from a subject with a compound that binds to and activates FgfR2b, and then returning the contacted cells to the subject. Ex vivo methods are particularly suitable when the cell to which the compound is to be delivered can easily be removed from and returned to the subject. In vitro and ex vivo routes of administration of a composition to a culture of cells can be accomplished by a method including, but not limited to, transfection, transformation, electroporation, microinjection, lipofection, adsorption, protoplast fusion, use of protein carrying agents, use of ion carrying agents, use of detergents for cell permeabilization, and simply mixing (e.g., combining) a compound in culture with a target cell.

Aerosol (inhalation) delivery can be performed using methods standard in the art (see, for example, Stribling et al., Proc. Natl. Acad. Sci. USA 189:11277-11281, 1992, which is incorporated herein by reference in its entirety). Oral delivery can be performed by complexing a therapeutic composition of the present invention to a carrier capable of withstanding degradation by digestive enzymes in the gut of an animal. Examples of such carriers include plastic capsules or tablets, such as those known in the art. Such routes can include the use of pharmaceutically acceptable carriers as described in more detail below. In a preferred embodiment the administration of the compound is by inhalation.

According to the present invention, administration of a compound useful in the present method activates FgfR2b in the lungs of a mammal.

According to the method of the present invention, an effective amount of a compound that binds FgfR2b and activates LESC proliferation (also referred to simply as “a compound”) to administer to a mammal comprises an amount that is capable of increasing LESCs proliferation without being toxic to the mammal. An amount that is toxic to a mammal comprises any amount that causes damage to the structure or function of a mammal (i.e., poisonous).

In one embodiment of the present invention, in a mammal that has lung injury, an effective amount of a compound to administer to a mammal is an amount that measurably increases proliferation of LESCs in the mammal as compared to prior to administration of the compound. In another embodiment, an effective amount of a compound to administer to a mammal is an amount that measurably increases proliferation of LESCs in the mammal as compared to a level of LESCs in a population of mammals with lung injury wherein the compound was not administered. The compound that binds to and activates FgfR2b according to the present invention is preferably capable of increasing LESCs proliferation in a mammal, even when the compound is administered after the onset of the physical symptoms of lung injury. Most preferably, an effective amount of the compound is an amount that reduces the symptoms of lung injury to the point where lung injury is no longer detected in the subject. In another embodiment, an effective amount of the compound is an amount that prevents, or substantially inhibits the onset of lung injury when the compound is administered prior to exposure of a provoking stimulus of lung injury such as exposure of the subject to environmental toxins, bacteria or viruses in a manner sufficient to induce lung injury in the absence of the compound.

A suitable single dose of a compound of the present invention to administer to a mammal is a dose that is capable of increasing LESCs proliferation in a mammal when administered one or more times over a suitable time period. In particular, a suitable single dose of a compound comprises a dose that increases proliferation of LESCs in mammals. A preferred single dose of a compound comprises between about 0.01 microgram×kilogram⁻¹ and about 10 milligram×kilogram⁻¹ body weight of a mammal. A more preferred single dose of a compound comprises between about 0.1 μg×kilogram⁻¹ and about 20 μg×kilogram⁻¹ body weight of said mammal. Another preferred single dose of a compound comprises between about 0.1 μg×kilogram⁻¹ and about 10 μg×kilogram⁻¹ body weight of said mammal. Another preferred single dose of a compound comprises between about 0.1 μg×kilogram⁻¹ and about 5 μg×kilogram⁻¹ body weight of said mammal. Another preferred single dose of a compound comprises between about 1 microgram×kilogram⁻¹ and about 10 milligram×kilogram⁻¹ body weight of a mammal. Another preferred single dose of a compound comprises between about 5 microgram×kilogram⁻¹ and about 7 milligram×kilogram⁻¹ body weight of a mammal. Another preferred single dose of a compound comprises between about 10 microgram×kilogram⁻¹ and about 5 milligram×kilogram⁻¹ body weight of a mammal. If the compound is delivered by aerosol or parenterally, a particularly preferred single dose of a compound comprises between about 0.01 microgram×kilogram⁻¹ and about 10 milligram×kilogram⁻¹ body weight of a mammal, and more preferably between about 0.01 milligram×kilogram⁻¹ and about 5 milligram×kilogram⁻¹ body weight of a mammal, and preferably between about 0.01 milligram×kilogram⁻¹ and about 1 milligram×kilogram⁻¹ body weight of a mammal, and more preferably between about 0.1 μg×kilogram⁻¹ and about 20 μg×kilogram⁻¹ body weight of said mammal, and more preferably, between about 0.1 μg×kilogram⁻¹ and about 10 μg×kilogram⁻¹ body weight of said mammal, and more preferably, between about 0.1 μg×kilogram⁻¹ and about 5 μg×kilogram⁻¹ body weight of said mammal. Typically, the compound can be administered in smaller doses when delivered by aerosol, as compared to other routes of delivery.

One of skill in the art will be able to determine that the number of doses of a compound to be administered to a mammal is dependent upon the extent lung injury and the underlying condition of which lung injury is a symptom, and the response of an individual subject to the treatment. In addition, the clinician will be able to determine the appropriate timing for delivery of the compound in a manner effective to reduce lung injury in the mammal. Preferably, the compound is delivered within 48 hours prior to exposure of the patient to an amount of a lung injury provoking stimulus (environmental toxin, bacteria, virus) effective to induce lung injury, and more preferably, within 36 hours, and more preferably within 24 hours, and more preferably within 12 hours, and more preferably within 6 hours, 5 hours, 4 hours, 3 hours, 2 hours, or 1 hour of prior to exposure of the subject to an amount of lung injury provoking stimulus effective to induce lung injury. In one embodiment, the compound is administered as soon as it is recognized (i.e., immediately) by the subject or clinician that the subject has been exposed or is about to be exposed to a lung injury provoking stimulus, and especially a lung injury provoking stimulus to which the patient is sensitized. In another embodiment, the compound is administered upon the first sign of development of lung injury, and preferably, within at least 2 hours of the development of symptoms of lung injury, and more preferably, within at least 1 hour, and more preferably within at least 30 minutes, and more preferably within at least 10 minutes, and more preferably within at least 5 minutes of development of symptoms of lung injury. Symptoms of lung injury in a subject include but are not limited to coughing, wheezing, chest pain, bloody sputm, shortness of breath, and fever. The subject can experience a single symptom as well as multiple symptoms.

Lung injury can be measured by a stress test that comprises measuring a mammal's respiratory system function in response to a provoking agent (i.e., stimulus). Lung injury can be measured as a change in respiratory function from baseline plotted against the dose of a provoking agent. Respiratory function can be measured by, for example, spirometry, plethysmograph, peak flows, symptom scores, physical signs (i.e., respiratory rate), wheezing, exercise tolerance, use of rescue medication (i.e., bronchodilators), cough and blood gases. In mice, lung function can be measured using a mechanical ventilation system such as a flexiVent system. In humans, spirometry can be used to gauge the change in respiratory function in conjunction with a provoking agent, such as an environmental toxin. In humans, spirometry is performed by asking a person to take a deep breath and blow, as long, as hard and as fast as possible into a gauge that measures airflow and volume. The volume of air expired in the first second is known as forced expiratory volume (FEV₁) and the total amount of air expired is known as the forced vital capacity (FVC). In humans, normal predicted FEV₁ and FVC are available and standardized according to weight, height, sex and race. An individual free of disease has an FEV₁ and a FVC of at least about 75% to 80% of normal predicted values for a particular person and a ratio of FEV₁/FVC of at least about 75% to 80%. Values are determined before (i.e, representing a mammal's resting state) and after (i.e., representing a mammal's higher lung resistance state) inhalation of the provoking agent. The position of the resulting curve indicates the sensitivity of the airways to the provoking agent.

The effect of increasing doses or concentrations of the provoking agent on lung function is determined by measuring the forced expired volume in 1 second (FEV₁) and FEV₁ over forced vital capacity (FEV₁/FVC ratio) of the mammal challenged with the provoking agent. FEV₁ and FVC values can be measured using methods known to those of skill in the art.

In another embodiment, the methods of the present invention improve a mammal's FEV₁ by at least about 5%, and more preferably by between about 6% and about 100%, more preferably by between about 7% and about 100%, and even more preferably by between about 8% and about 100% of the mammal's predicted FEV₁. In another embodiment, the method of the present invention improves a mammal's FEV₁ by at least about 5%, and preferably, at least about 10%, and even more preferably, at least about 25%, and even more preferably, at least about 50%, and even more preferably, at least about 75%.

In a preferred embodiment of the present invention, spirometry is used to detect and/or measure lung injury in a subject.

In another embodiment, the compound administration to a subject are given once every 1-2 hours until signs of reduction of lung injury appear, and then as needed until the symptoms of lung injury are gone. In one embodiment, the compound of the present invention can be administered on a regular basis as a prophylactic treatment for the prevention of lung injury, or minimally, to reduce the risk of developing lung injury. Prophylactic administration protocols can be developed by the clinician and will depend on the dosage and general need and health of the individual patient, but generally, administration every 1-7 days is contemplated as being sufficient to inhibit lung injury in the individual.

Another embodiment of the present invention is a method to treat a condition related to injury of lung epithelial cells in a subject. This method includes isolating cells, such as LESCs, that are expressing FGfR2b from a donor. The cells expressing FgfR2b can be isolated from a cellular sample obtained from the donor by means of a lung biopsy. The donor can be the subject, a cadaver or another individual, such as a healthy donor or a person suffering from a disease such as cystic fibrosis. Cells from cystic fibrosis patients can be subjected to homologous recombination to correct the defective CFTR gene.

To isolate the cells expressing FgfR2b from a cellular sample, an antibody that specifically binds FgfR2b can be used to label the FgfR2b expressing cells. The antibody can be labeled with any known label such as a fluorescent label or a radioactive label.

In this embodiment, the isolated FgfR2b expressing cells are cultured to multiply the cells. The step of culturing can be conducted under appropriate conditions for the cells to multiply. Cells can be cultured in the presence of 500 ng/ml Fgf10 in DMEM/F12+10% FBS in dishes coated with matrigel or irradiated MEFs (murine embryonic fibroblasts) or proper substitute. Optionally, during the step of culturing, the cells can be exposed to or contacted with a compound that binds FgfR2b (as described above), and the exposure to the compound can lead to proliferation of the cells. After the step of culturing the cells are introduced in the subject. Introduction of the cultured cells can be by intratracheal inhalation, intravenous administration, as well as by intranasal administration. In one embodiment, introduction of the cells to the subject repairs lung injury.

In one embodiment, recombinant FgfR2b is expressed by a cell (i.e., a cell-based assay). In another embodiment a recombinant FgfR2b is in a cell lysate, or is purified or produced free of cells (e.g., a soluble FgfR2b). In accordance with the present invention, a cell-based assay is conducted under conditions which are effective to screen for regulatory compounds useful in the method of the present invention. Effective conditions include, but are not limited to, appropriate media, temperature, pH and oxygen conditions that permit cell growth. An appropriate, or effective, medium refers to any medium in which a cell of the present invention, when cultured, is capable of cell growth and expression of recombinant FgfR2b. Such a medium is typically a solid or liquid medium comprising growth factors and assimilable carbon, nitrogen and phosphate sources, as well as appropriate salts, minerals, metals and other nutrients, such as vitamins. Culturing is carried out at a temperature, pH and oxygen content appropriate for the cell. Such culturing conditions are within the expertise of one of ordinary skill in the art.

According to the present invention, a host cell can be transfected in vivo (i.e., by delivery of the nucleic acid molecule into a mammal), ex vivo (i.e., outside of a mammal for reintroduction into the mammal, such as by introducing a nucleic acid molecule into a cell which has been removed from a mammal in tissue culture, followed by reintroduction of the cell into the mammal); or in vitro (i.e., outside of a mammal, such as in tissue culture for production of a recombinant Fgf, a fragment or a homologue thereof). Transfection of a nucleic acid molecule into a host cell can be accomplished by any method by which a nucleic acid molecule can be inserted into the cell. Transfection techniques include, but are not limited to, transfection, electroporation, microinjection, lipofection, adsorption, and protoplast fusion. Preferred methods to transfect host cells in vivo include lipofection, viral vector delivery and adsorption.

A recombinant cell of the present invention comprises a host cell transfected with a nucleic acid molecule that encodes Fgf, a fragment or a homologue thereof. It may be appreciated by one skilled in the art that use of recombinant DNA technologies can improve expression of transfected nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules encoding Fgf, a fragment or a homologue thereof include, but are not limited to, operatively linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules to correspond to the codon usage of the host cell, and deletion of sequences that destabilize transcripts. The activity of an expressed recombinant Fgf or a homologue thereof may be improved by fragmenting, modifying, or derivatizing nucleic acid molecules encoding such a protein.

A further embodiment of the present invention is a method to detect lung injury in a subject. This method comprises detecting the level of expression of FgfR2b in a subject sample, wherein an elevated level of expression of FgfR2b in the subject sample is indicative of lung injury. The determination of whether a level of expression of FgfR2b is elevated can be determined in a variety of ways. For example, the detected level can be compared to known levels for normal (i.e., non-injured) tissue or cells. Alternatively, the detected level can be compared to the level detected in control individuals or to known levels of a sample from the subject taken at time when no injury was present. The level of expression of FgfR2b can be determined by fluorescence activated cell sorting (FACS), with an antibody to FgfR2b linked to a fluorescent dye.

Another further embodiment of the present invention is a pharmaceutical composition comprising a compound that binds FgfR2b, as such compounds are described above, and a pharmaceutically acceptable carrier. As used herein a pharmaceutical acceptable carrier refers to any substance suitable for delivering a pharmaceutical composition useful in the methods of the present invention. A pharmaceutical composition of the present invention generally includes a carrier, and preferably, a pharmaceutically acceptable carrier. According to the present invention, a “pharmaceutically acceptable carrier” includes pharmaceutically acceptable excipients and/or pharmaceutically acceptable delivery vehicles, which are suitable for use in administration of the composition to a suitable in vitro, ex vivo or in vivo site. A suitable in vitro, in vivo or ex vivo site is preferably a LESC that expresses FgfR2b. Preferred pharmaceutically acceptable carriers are capable of maintaining a protein, compound, or nucleic acid molecule according to the present invention in a form that, upon arrival of the protein, compound, or nucleic acid molecule at the cell target in a culture or in patient, the protein, compound or nucleic acid molecule is capable of interacting with its target (e.g., a naturally occurring FgfR2b).

Suitable excipients of the present invention include excipients or formularies that transport or help transport, but do not specifically target a composition to a cell (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- or o-cresol, formalin and benzol alcohol. Compositions of the present invention can be sterilized by conventional methods and/or lyophilized.

One type of pharmaceutically acceptable carrier includes a controlled release formulation that is capable of slowly releasing a composition of the present invention into a patient or culture. As used herein, a controlled release formulation comprises a compound of the present invention (e.g., a protein (including homologues), a drug, an antibody, a nucleic acid molecule, or a mimetic) in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other carriers of the present invention include liquids that, upon administration to a patient, form a solid or a gel in situ. Preferred carriers are also biodegradable (i.e., bioerodible). Natural lipid-containing delivery vehicles include cells and cellular membranes. Artificial lipid-containing delivery vehicles include liposomes and micelles. A delivery vehicle of the present invention can be modified to target to a particular site in a patient, thereby targeting and making use of a compound of the present invention at that site. Suitable modifications include manipulating the chemical formula of the lipid portion of the delivery vehicle and/or introducing into the vehicle a targeting agent capable of specifically targeting a delivery vehicle to a preferred site, for example, a preferred cell type. Other suitable delivery vehicles include gold particles, poly-L-lysine/DNA-molecular conjugates, and artificial chromosomes.

Other suitable carriers include any carrier that can be bound to or incorporated with the compound that extends that half-life of the compound to be delivered. Such a carrier can include any suitable protein carrier or even a fusion segment that extends the half-life of a compound that binds FgfR2b.

A pharmaceutically acceptable carrier which is capable of targeting is herein referred to as a “delivery vehicle.” Delivery vehicles of the present invention are capable of delivering a formulation, including an FgfR2b activating agent to a target site in a mammal. A “target site” refers to a site in a mammal to which one desires to deliver a therapeutic formulation. For example, a target site can be any cell which is targeted by direct injection or delivery using liposomes or antibodies, such as bi-valent antibodies. A delivery vehicle of the present invention can be modified to target to a particular site in a mammal, thereby targeting and making use of the agent complexed with the liposome at that site. Suitable modifications include manipulating the chemical formula of the lipid portion of the delivery vehicle and/or introducing into the vehicle a compound capable of specifically targeting a delivery vehicle to a preferred site, for example, a preferred cell type. Specifically, targeting refers to causing a delivery vehicle to bind to a particular cell by the interaction of the compound in the vehicle to a molecule on the surface of the cell. Suitable targeting compounds include ligands capable of selectively (i.e., specifically) binding another molecule at a particular site. Examples of such ligands include antibodies, antigens, receptors and receptor ligands. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics. The compound used in the present method can be in any form suitable for delivery, including, but not limited to, a liquid, an aerosol, a capsule, a tablet, a pill, a powder, a gel and a granule. Preparations of agents that are particularly suitable for parenteral administration include sterile aqueous or nonaqueous solutions, suspensions or emulsions. Examples of nonaqueous solvents or vehicles are propylene glycol, polyethylene glycol, vegetable oils such as olive oil and injectable organic esters such as ethyl oleate.

In solid dosage forms, the agent can be admixed with at least one inert diluent such as sucrose, lactose or starch. Such dosage forms can also comprise, as is normal practice, additional substances other than inert diluent. In the case of capsules, tablets, and pills, the dosage forms may also comprise buffering agents, pH-sensitive polymers, or any other slow-releasing encapsulants (i.e., controlled release vehicles) which are typically used as encapsulating compositions in the food and drug industry or any other controlled release formulations. Tablets and pills can additionally be prepared with an enteric coating.

Liquid dosage forms of agent for oral administration include pharmaceutically acceptable emulsions, solutions, suspensions, syrups and elixirs, containing inert diluents commonly used in the pharmaceutical art. Besides inert diluents, compositions can also include wetting agents, emulsifying, and suspending, and sweetening agents.

Isolated nucleic acid molecules to be administered in a method of the present invention include: (a) isolated nucleic acid molecules useful in the method of the present invention in a non-targeting carrier (e.g., as “naked” DNA molecules, such as is taught, for example in Wolff et al., 1990, Science 247, 1465-1468); and (b) isolated nucleic acid molecules of the present invention complexed to a delivery vehicle of the present invention. Particularly suitable delivery vehicles for local administration of nucleic acid molecules comprise liposomes, viral vectors and ribozymes. Delivery vehicles for local administration can further comprise ligands for targeting the vehicle to a particular site. One preferred delivery vehicle of the present invention is a liposome. A liposome is capable of remaining stable in an animal for a sufficient amount of time to deliver a nucleic acid molecule described in the present invention to a preferred site in the animal. A liposome, according to the present invention, comprises a lipid composition that is capable of delivering a nucleic acid molecule described in the present invention to a particular, or selected, site in a mammal. A liposome according to the present invention comprises a lipid composition that is capable of fusing with the plasma membrane of the targeted cell to deliver a nucleic acid molecule into a cell. Suitable liposomes for use with the present invention include any liposome. Preferred liposomes of the present invention include those liposomes typically used in, for example, gene delivery methods known to those of skill in the art. More preferred liposomes comprise liposomes having a polycationic lipid composition and/or liposomes having a cholesterol backbone conjugated to polyethylene glycol.

A liposome comprises a lipid composition that is capable of fusing with the plasma membrane of the targeted cell to deliver a nucleic acid molecule or other agent (e.g., a peptide) into a cell. Preferably, the transfection efficiency of a liposome is at least about 0.5 microgram (m) of DNA per 16 nanomole (nmol) of liposome delivered to about 10⁶ cells, more preferably at least about 1.0 μg of DNA per 16 nmol of liposome delivered to about 10⁶ cells, and even more preferably at least about 2.0 μg of DNA per 16 nmol of liposome delivered to about 10⁶ cells. A preferred liposome is between about 100 and about 500 nanometers (nm), more preferably between about 150 and about 450 nm and even more preferably between about 200 and about 400 nm in diameter.

Complexing a liposome with a nucleic acid molecule or other agent of the present invention can be achieved using methods standard in the art. A suitable concentration of a nucleic acid molecule or other agent to add to a liposome includes a concentration effective for delivering a sufficient amount of nucleic acid molecule and/or other agent to a cell such that the biological activity of the FgfR2b is increased in a desired manner. Preferably, nucleic acid molecules are combined with liposomes at a ratio of from about 0.1 μg to about 10 μg of nucleic acid molecule of the present invention per about 8 nmol liposomes, more preferably from about 0.5 μg to about 5 μg of nucleic acid molecule per about 8 nmol liposomes, and even more preferably about 1.0 μg of nucleic acid molecule per about 8 nmol liposomes.

Another preferred delivery vehicle comprises a viral vector. A viral vector includes an isolated nucleic acid molecule useful in the method of the present invention, in which the nucleic acid molecules are packaged in a viral coat that allows entrance of DNA into a cell. A number of viral vectors can be used, including, but not limited to, those based on alphaviruses, poxviruses, adenoviruses, herpesviruses, lentiviruses, adeno-associated viruses and retroviruses.

Also included in the present invention are therapeutic molecules known as ribozymes. A ribozyme typically contains stretches of complementary RNA bases that can base-pair with a target RNA ligand, including the RNA molecule itself, giving rise to an active site of defined structure that can cleave the bound RNA molecule (See Maulik et al., 1997, supra). Therefore, a ribozyme can serve as a targeting delivery vehicle for the nucleic acid molecule.

Another embodiment of the present invention is a method to treat a condition related to proliferation of lung epithelial cells in a subject by administering a compound that binds to FgfR2b to block receptor signaling. In a further embodiment, the receptor signaling is blocked by preventing dimerization of FgfR2b to occur. An example of a compound that binds to FgfR2B and blocks receptor signaling is an antibody. In a preferred embodiment, the compound comprises a cytotoxic agent. In a further embodiment the compound is an antibody that recognizes FgfR2b and delivers it to a cytotoxic agent that specifically kill cells that express FgfR2b thus killing the stem cells that can proliferate and form tumors. Examples of cytotoxic agents that are useful in antibody-cytotoxic agent conjugates (ACCs) of the present invention include but are not limited to calicheamicin and related chemotherapy agents as well as diphtheria toxin, ricin toxin, abrin toxin, pseudomonas exotoxin, shiga toxin, α-amanitin, pokeweed antiviral protein (PAP), ribosome inhibiting proteins (RIP), especially the ribosome inhibiting proteins of barley, wheat, flax, corn, rye, gelonin, abrin, modeccin and certain cytotoxic chemicals such as, for example, melphalan, methotrexate, nitrogen mustard, doxorubicin and daunomycin. Some of these toxins (e.g., bacterial toxins and certain plant toxins) can be characterized by whether or not a “whole” molecule of a given toxin is employed. The term “whole” may be taken to mean that the molecule has at least a toxic domain, a translocational domain and a cell binding domain. If, however, one or more of these domains are removed from a “whole” toxin molecule, then the resulting molecule will be characterized as a “modified” toxin or “modified” molecule of that toxin. Some of these toxin types (e.g., bacterial and plant toxins) also can be further characterized by their possession of so-called “A-chain” and “B-chain” groups in their molecular structures. It also should be noted that the toxic domain is often referred to as the “A-chain” portion of the toxin molecule while the toxic domain, translocation domain and cell-binding domain are often collectively referred to as the “whole” toxin or the A-chain plus the B-chain molecules.

Another embodiment of the present invention relates to a method to identify a compound for increasing proliferation of LESCs in a mammal. The method includes the steps of: (a) contacting FgfR2b with a putative regulatory compound; (b) detecting whether the putative regulatory compound binds to FgfR2b; and (c) administering a putative regulatory compound which binds to FgfR2b to a non-human test mammal in which lung injury can be induced, and detecting whether the putative regulatory compound increase proliferation of LESCs in the test mammal in the presence of the compound as compared to in the absence of the putative regulatory compound. Putative regulatory compounds that bind to FgfR2b and that increase LESCs proliferation in the test mammal are identified as compounds which reduce lung injury. In a preferred embodiment, step (c) of administering comprises administering the putative regulatory compound which binds to FgfR2b to a non-human test mammal that has been sensitized to a toxin (such as naphthalene) and detecting whether the putative regulatory compound causes proliferation of LESCs in the test mammal when the mammal is challenged with the toxin, as compared to in the absence of the putative regulatory compound. Putative regulatory compounds that bind to FgfR2b and increase proliferation of LESCs in the test mammal are identified as compounds which reduce lung injury.

As used herein, the term “putative” refers to compounds having an unknown or previously unappreciated regulatory activity in a particular process. As such, the term “identify” is intended to include all compounds, the usefulness of which as a regulatory compound of FgfR2b activation for the purposes of increasing proliferation of LESCs is determined by a method of the present invention.

The following examples are provided for illustrative purposes, and are not intended to limit the scope of the invention as claimed herein. Any variations which occur to the skilled artisan are intended to fall within the scope of the present invention. All references cited in the present application are incorporated by reference herein to the extent that there is no inconsistency with the present disclosure.

### EXAMPLES

### Example 1

### Example 1A

### Mouse Strains

Tet-Dkk1 mice were generated by knocking in a Tet-Dkk1 cassette in the hypoxanthine-guanine phosphoribosyltransferase (Hprt) locus through homologous recombination. CMV-Cre mice c-Tg(CMV-cre)1Cgn/J (Jackson Laboratories (JAX)) were crossed with Rosa26-rtTAflox mice (Belteki, G., Haigh, J., Kabacs, N., Haigh, K., Sison, K., Costantini, F., Whitsett, J., Quaggin, S. E. and Nagy, A. (2005) Conditional and inducible transgene expression in mice through the combinatorial use of Cre-mediated recombination and tetracycline induction. Nucleic Acids Res 33, e51) to generate Rosa26-rtTA mice expressing rtTA from the Rosa26 promoter in every single cell of the body. Rosa26-rtTA mice were crossed with Tet-sFgfr2b (“s” indicates soluble/secreted) mice (Hokuto, I., Perl, A. K. and Whitsett, J. A. (2003). Prenatal, but not postnatal, inhibition of fibroblast growth factor receptor signaling causes emphysema. J Biol Chem 278, 415-21), Tet-Fgf10 (Clark, J. C., Tichelaar, J. W., Wert, S. E., Itoh, N., Perl, A. K., Stahlman, M. T. and Whitsett, J. A. (2001). FGF-10 disrupts lung morphogenesis and causes pulmonary adenomas in vivo. Am J Physiol Lung Cell Mol Physiol 280, L705-15) and Tet-Dkk1 to generate double transgenic mice. These mice are on a mixed genetic background and allow inducible expression of sFgfr2b, Fgf10 and Dkk1 simply by feeding the mice with doxycycline containing food (Rodent diet with 625 mg/kg Doxycycline, Harlan Teklad TD.09761). TOPGAL mice are described in DasGupta, R., and Fuchs, E. (1999) Multiple roles for activated LEF/TCF transcription complexes during hair follicle development and differentiation Development 126, 4557-4568. smMHC-Cre (Tg(Myh11-cre,-EGFP)2Mik/J), Rosa26R-LacZ (Gt(Rosa)26Sortm1Sor), Rosa26R-eYPF (Gt(Rosa)26Sortm1(eYFP)Cos); Rosa26-NotchICD (Tg(CAG-Bgeo,-NOTCH1,-EGFP)1Lbe/J), Scgba1a1-rtTA (Tg(Scgba1a1-rtTA)1Jaw/J) and Tet-O-Cre (Tg(tetO-cre)1Jaw/J) mice were obtained from TAX. Adult mice were 8 weeks old at time of naphthalene administration.

### Example 1B

### β-Galactosidase Staining

Tissues containing Rosa26R, TOPGAL or Fgf10^{LacZ} alleles were dissected and β-galactosidase staining was performed at different stages post-naphthalene injury (3 days, 7 days, 14 days and 21 days). Lungs were dissected and fixed in 4% paraformaldehyde (PFA) in PBS at room temperature for 5 min, rinsed in PBS, then injected with freshly prepared X-gal solution, transferred into a vial of X-gal solution and stained at 37° C. overnight. After rinsing with PBS, lungs were post-fixed in 4% PFA in PBS at room temperature overnight. For microtome sections, after 4% PFA fixation, lungs were washed in PBS, dehydrated, and paraffin embedded. For clearing, after 4% PFA fixation, lungs were washed in PBS, dehydrated and cleared with BABB (1:2 Benzyl Alcohol and Benzyl Benzoate) as follows: tissue transferred to 1:2 BABB and ethanol for 20 min, 2:1 BABB and ethanol for 20 min, and 100% BABB for 20 min.

### Example 1C

### Immunohistochemistry (IHC) and Fluorescence

All staining was done on paraffin sections of formalin fixed lungs. IHC was performed with the Histostain-Plus Kit (Invitrogen). IHC and fluorescent staining was performed with the following primary antibodies:

TABLE 1
IHC and Fluorescent Staining Primary Antibodies

| Primary Antibody | Host | Dilution | Manufacturer |
|---|---|---|---|
| β-tubulin | Mouse | 500 | Seven Hills Bioreagents |
| (clone 3F3-G2) | | | |
| Wnt-7b | Goat | 50 | R&D Systems |
| SCGB1A1 (T-18) | Goat | 200 | Santa Cruz Biotechnology, Inc. |
| SCGB1A1 | Rabbit | 500 | Seven Hills Bioreagents |
| CGRP | Rabbit | 5000 | Sigma-Aldrich |
| CGRP | Chicken | 75 | Neuromics |
| Bek (C-17) (Fgfr2b) | Rabbit | 200 | Santa Cruz Biotechnology, Inc. |
| Activated Notch1 | Rabbit | 250 | AbCam |
| Snail (E-18) | Goat | 50 | Santa Cruz Biotechnology, Inc. |
| α-SMA Cy3 | mouse | 200 | Sigma-Aldrich |
| conjugate | | | |
| GFP | Chicken | 500 | Ayes Labs, Inc. |
| N-Terminal Pro | Rabbit | 500 | Seven Hills Bioreagents |
| Sp-C | | | |
| β-catenin Ser552 | Rabbit | 200 | Cell Signaling Technology |

All fluorescent staining was performed with secondary's from Jackson Immunoresearch (except the Cy3 conjugated α-SMA) and mounted using Vectashield ® with DAPI (Vector Labs).

### Example 1D

### qPCR

RNA was isolated from lung accessory lobes using RNALater (Ambion) and Total RNA Kit I (Omega Biotek) according to manufacturer's instructions. RNA concentration was determined by spectrophotometry. cDNA was generated using SuperScript® III First-Strand Synthesis System (Invitrogen) according to manufacturer's instructions. Comparative Real Time PCR was performed for β-glucuronidase, SCGB1A1, and Wnt-7b Taqman® Gene Expression Assays (Applied Biosystems) using a StepOne Plus system (Applied Biosystems). β-glucuronidase was used as a reference control to normalize equal loading of template cDNA.

### Example 1E

### Naphthalene Treatment

Naphthalene (Sigma-Aldrich) was dissolved in corn oil at 30 mg/mL, and administered intraperitoneal at 8 weeks of age with doses adjusted according to strain to achieve a 95% decrease in the abundance of Scgb1a1mRNA in total lung RNA of wild-type.

### Example 1F

### FACS Analysis

Cells were prepared from non-injured control mice, 7 days post-naphthalene injury control mice, and 7 days post-naphthalene injury Rosa26-rtTA;Tet-Fgf10 mice using Collagenase type 2 (Worthington) and Red Blood Cell Lysis Buffer (eBioscience). Live/dead staining was performed with LIVE/DEAD® Fixable Violet Dead Cell Stain Kit (Invitrogen). Cells were fixed and permeabilized with Perm/Wash Buffer and Cytofix/Cytoperm (BD). Cells were then stained with Sp-C (Seven Hills Bioreagents), Fgfr2b (Bek C-(Santa Cruz Biotechnology, Inc.) or SCGB1A1 (Santa Cruz Biotechnology, Inc.) and labeled with Zenon® Alexa Fluor® Labeling Kits (Invitrogen).

### Example 1G

### Proliferation Analysis

Mice were given intraperitoneal injections of 104 of bromodeoxyuridine (BrdU, GE Healthcare) per gram body weight 4 hours before sacrifice. Lungs were fixed in 4% paraformaldehyde, dehydrated, and paraffin embedded. Sections were treated with monoclonal anti-bromodeoxyuridine (Clone BU-1, GE Healthcare), following manufacturer's instructions. FITC-labeled anti-mouse secondary antibodies were used (Jackson Immunoresearch). All slides were mounted using Vectashield® with DAPI.

### Example 2

### Example 2A

This example illustrates that after injury of lung epithelial cells by exposure to an environmental toxin (naphthalene), PMSCs undergo proliferation. Using a 4 hour BrdU pulse labeling assay, mice were administered intraperitoneal injections of bromodeoxyuridine per gram body weight four hours before sacrifice. Proliferation of PSMSc was assayed. BrdU labeling of lungs 3 days after injury, indicated a robust proliferation of the PSMCs compared to no proliferation in corn oil treated lungs (control mice with no exposure to naphthalene). This proliferation was blocked in mice overexpressing Dickkopf-1 (Dkk1), which is known to inhibit the Wnt signaling.

### Example 2B

This example illustrates that after injury of lung epithelial cells by exposure to naphthalene, cells undergo active β-catenin signaling. A TOPGAL mice reporter line (DasGupta, R., and Fuchs, E. (1999) Multiple roles for activated LEF/TCF transcription complexes during hair follicle development and differentiation Development 126, 4557-4568) which consists of a beta-galactosidase gene driven by a T-cell factor β-catenin responsive promoter, was used. During normal homeostasis in 2 month old mice the majority of Clara cells were TOPGAL positive demonstrating that these cells are undergoing active β-catenin signaling. No TOPGAL activity was detected in the PSMCs during normal homeostasis in 2 month old adult lungs. When TOPGAL mice were injured with naphthalene, 3 days after challenge, the majority of Clara cells were wiped out except for the BASCs at the bronchio-alveolar duct junctions (BADJs), which showed a strong induction in TOPGAL activity. Clara^{V} cells adjacent to neuroendocrine bodies (NEBs) were also TOPGAL positive. TOPGAL activation was also determined in the PSMCs surrounding the injured airways, indicating a reactivation and potential dedifferentiation of these cells. To demonstrate that TOPGAL activity in the PSMCs as well as the BASCs and Clara^{V} cells is Wnt ligand dependent the TOPGAL mice were crossed with Rosa-rtTA; Tet-Dkk1 mice. Overexpression of Dkk1 prevented the induction of TOPGAL activity in the PSMCs as well as the BASCs 3 days after injury of lung epithelial cells by exposure to naphthalene.

### Example 2C

This example illustrates that induction of Fgf10 occurs after injury of lung epithelial cell. Fgf10 expression was analyzed using Fgf10^{LacZ} (Kelly, R. G., Brown, N. A., and Buckingham, M. E. (2001) The arterial pole of the mouse heart forms from Fgf10-expressing cells in pharyngeal mesoderm. Dev Cell 1, 435-440; Mailleux, A. A., Kelly, R., Veltmaat, J. M., De Langhe, S. P., Zaffran, S., Thiery, J. P., and Bellusci, S. (2005) Fgf10 expression identifies parabronchial smooth muscle cell progenitors and is required for their entry into the smooth muscle cell lineage. Development 132, 2157) mice in 2 month old adult lungs during normal homeostasis, 3 days after injury of lung epithelial cells from exposure to naphthalene, as well as 3 days after injury of lung epithelial cells from exposure to naphthalene while overexpressing Dkk1. PSMCs do not express Fgf10 during normal homeostasis, but showed a strong induction in Fgf10 expression 3 days after naphthalene injury. Overexpression of Dkk1 inhibited Fgf10 induction in PSMCs 3 days after injury.

### Example 2D

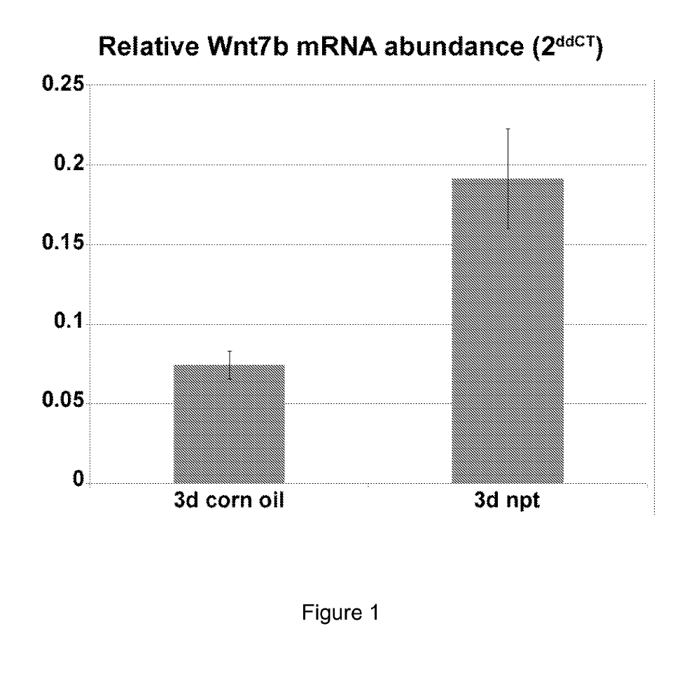

This example illustrates that Wnt7b expression is induced in PSMCs after injury of lung epithelial cells from exposure to naphthalene. 3 days after naphthalene injury, surviving airway ciliated cells showed a robust induction in Wnt7b expression, possibly activating surrounding PSMCs in a paracrine fashion (FIG. 1).

### Example 3

### Example 3A

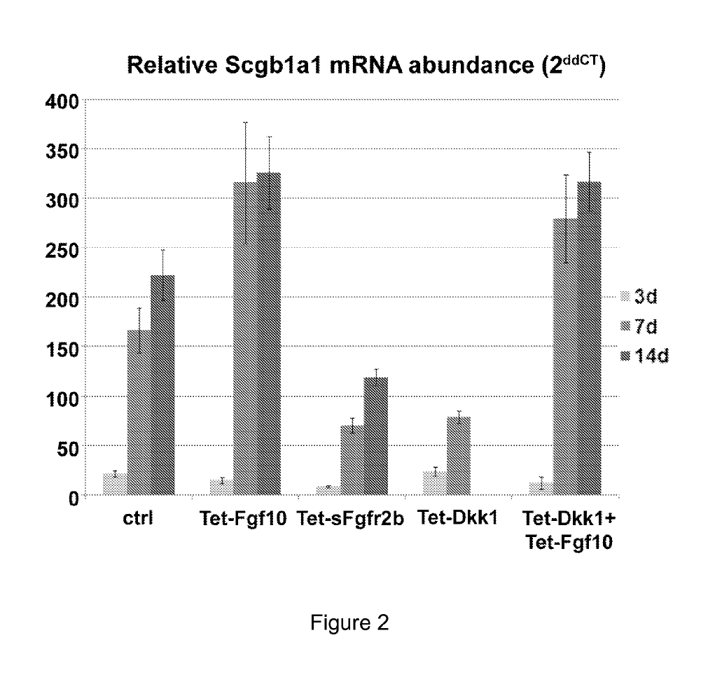

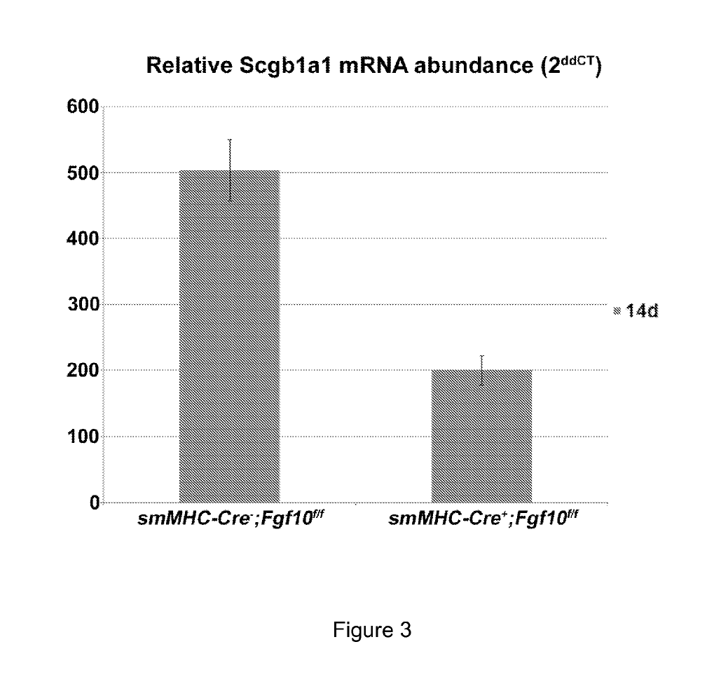

This examples illustrates the importance of the Wnt induced FGF10 secreted by the PSMCs, for epithelial regeneration after injury of lung epithelial cells from exposure to naphthalene. Naphthalene-injured 2 month old smMHC-cre;Fgf10^{f/f} mice were generated by conditionally deleting Fgf10 specifically from the smooth muscle cells, Rosa26-rtTA;Tet-sFgfr2b, Rosa26-rtTA;Tet-Fgf10, Rosa26-rtTA; Tet-Dkk1 and Rosa26-rtTa;Tet-Dkk1;Tet-Fgf10. These mice conditionally overexpressed a dominant negative soluble secreted Fgfr2b receptor, Fgf10, Dkk1, and both Dkk1 and FGF10 respectively. Inhibition of FGF10 signaling by overexpression of the dominant negative FgfR2b significantly inhibited Clara cell regeneration after naphthalene injury as quantified by real time PCR for Scgb1a1 (Secretoglobulin 1a1), which is specifically expressed by Clara cells (FIG. 2). Overexpression of Fgf10 significantly accelerated Clara cell regeneration whereas mice overexpressing Dkk1 showed a significant impairment in regeneration and did not survive up to 2 weeks after injury (FIG. 3). This impaired regeneration was rescued by coexpressing Dkk1 and Fgf10 together and these mice showed again an accelerated repair similar to mice overexpressing Fgf10 alone (FIG. 3). This result indicates that the main mechanism by which overexpression of Dkk1 impairs epithelial regeneration is by inhibiting Wnt7b induced Fgf10 expression in the PSMCs.

### Example 3B

This example illustrates that FGF10 affects both the amplification of the BASCs at the BADJs as well as variant Clara cells near the NEBs. To demonstrate that the PSMCs are indeed the source of FGF10, important for regeneration, smMHC-Cre;Fgf1^{f/f} mice were analyzed. Because these mice were on a different background and because they did not tolerate injury of lung epithelial cells from exposure to naphthalene very well, lower levels of injury were used to allow survival. Clara cell regeneration was shown to be severely impaired in mice exhibiting a smooth muscle cell specific deletion of Fgf10.

### Example 4

### Example 4A

Epithelial Wnt signaling has been shown to accelerate Clara cell regeneration after naphthalene injury and has been shown to amplify the BASCs at the BADJs (Reynolds, S. D., Zemke, A. C., Giangreco, A., Brockway, B. L., Teisanu, R. M., Drake, J. A., Mariani, T., Di, P. Y., Taketo, M. M., and Stripp, B. R. (2008) Conditional stabilization of beta-catenin expands the pool of lung stem cells Stem Cells 26, 1337-1346; and Zhang, Y., Goss, A. M., Cohen, E. D., Kadzik, R., Lepore, J. J., Muthukumaraswamy, K., Yang, J., DeMayo, F. J., Whitsett, J. A., Parmacek, M. S., et al. (2008) A Gata6-Wnt pathway required for epithelial stem cell development and airway regeneration. Nat Genet 40, 862-870). This example illustrates that FGF10 activates β-catenin signaling directly in the epithelium independent of Wnt ligands. The TOPGAL Wnt signaling reporter allele was crossed into the different mouse lines described previously. The mice were exposed to naphthalene and epithelial β-catenin signaling was monitored 7 days after injury. Seven days after injury, control TOPGAL mice were partially regenerated and showed TOPGAL activity in the regenerating Clara cells at both the BADJs as well as near the NEBs. Mice overexpressing Fgf10 showed a robust increase in Clara cell regeneration, as analyzed above, and showed a strong increase in epithelial TOPGAL activity in the Clara cells at both the BADJs and the NEBs. Mice overexpressing the secreted dominant negative Fgfr2b receptor or Dkk1 showed a strong decrease in Clara cell regeneration and had limited TOPGAL activity in the Clara cells at both the BADJs and NEBs. This indicates that inhibition of FGF10 signaling through either sequestering the FGF10 ligand or suppressing the expression of Fgf10, by sFgfR2b or DKK1 respectively, inhibits not only Clara cell regeneration but also epithelial β-catenin signaling. In addition, injured lungs from mice overexpressing both Dkk1 and Fgf10 showed a rescue in Clara cell regeneration as well as β-catenin signaling at both the BADJs and NEBs.

### Example 5

### Example 5A

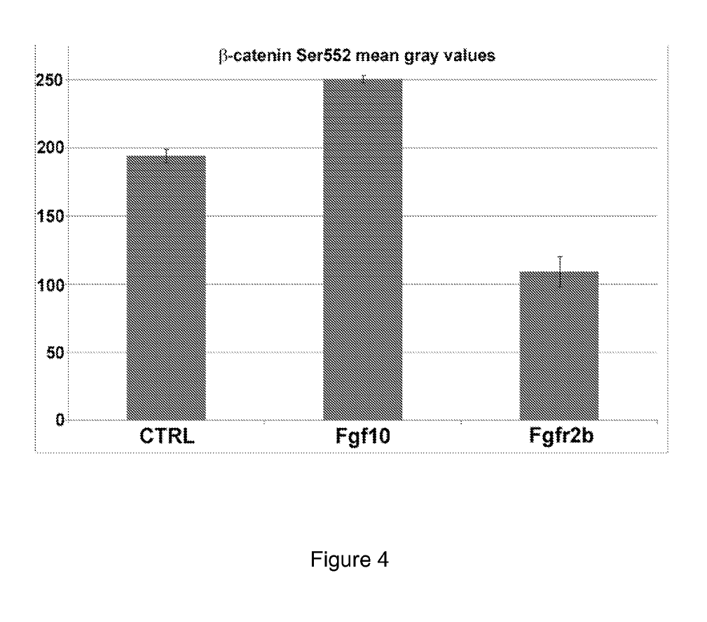

This example illustrates that FGF10 signaling induces AKT mediated phosphorylation of β-catenin, maintenance/amplification of variant Clara cells and that FgfR2b is an extracellular marker. The main mechanism through which FGF10 is thought to activate β-catenin signaling is by activation of the PI3K-AKT pathway. AKT is not only known to inhibit GSK3 β therefore preventing the degradation of β-catenin, but also to phosphorylate β-catenin directly on Ser552 to drive it to the nucleus. An immunostaining for p-β-catenin-Ser552 was performed on lung samples from control, Rosa26-rtTa;Tet-Fgf10 and Rosa26-rtTa-Tet-sFgr2b samples 14 days after injury of lung epithelial cells from exposure to naphthalene. Modest phosphorylation of β-catenin on Ser552 in control lungs was detected (“ctrl” in FIG. 4). This phosphorylation was increased in lungs overexpressing Fgf10 (“Tet-Fgf10” in FIG. 4) and was dramatically decreased in lungs overexpressing the secreted dominant negative Fgfr2b receptor (“Tet-sFgfr2b” in FIG. 4), indicating that FgfR2b can be used as an extracellular maker for Scgb1a1-Sftpc double positive airway epithelial stem cells. Fgfr2b expression is downstream of FGF10 as well as β-catenin signaling.

### Example 5B

This example illustrates that FGF10 is important in generating BASCs at the BADJs by activating epithelial β-catenin signaling. The effect of FGF10 on the amplification of the BASCs at the BADJs was analyzed by a double immunostaining for Scgb1a1 (BASCs cell marker) and Sftpc (BASCs cell marker) on control lungs (no injury) and Fgf10 overexpressing lungs 21 days after injury of lung epithelial cells from exposure to naphthalene. An increase in BASCs or Scgb1a1-Sftpc double positive cells is normally seen shortly after injury of lung epithelial cells from exposure to naphthalene. These double positive cells usually do not persist by 21 days after injury. Overexpression of FGF10 did not only amplify the number of BASCs (Scgb1a1-Sftpc double positive cells) after injury, but also maintained them as long as Fgf10 expression was induced. In addition, FGF10 was also able to generate these Scgb1a1-Sftpc double positive airway epithelial stem cells near the NEBs. A cluster of Sftpc positive Clara cells were detected adjacent to a NEB in an Fgf10 overexpressing 7 days after injury of lung epithelial cells from exposure to naphthalene.

### Example 6

This example illustrates that FgfR2b is a marker for airway epithelial stem cells in the adult lung after injury of lung epithelial cells from exposure to naphthalene. To quantify the effect of naphthalene injury and Fgf10 expression on the generation/amplification of Scgb1a1-Sftpc or Scgb1a1-Fgfr2b double positive cells, a FACS analysis for these markers on single cell whole lung digests from non-injured control lungs as well as control lung and Fgf10 overexpressing lungs 7 days after injury of lung epithelial cells from exposure to naphthalene. In non-injured control lungs, only a small fraction of the cells were Scgb1a1-Sftpc or Scgb1a1-Fgfr2b double positive, 0.7% versus 1.31% respectively. Control lungs that were injured showed a marked increase in Scgb1a1-Sftpc or Scgb1a1-Fgfr2b double positive airway epithelial stem cells 7 days after injury, 5.16% versus 9.34% respectively. Finally, Fgf10 overexpressing lungs showed an even bigger increase in Scgb1a1-Sftpc or Scgb1a1-Fgfr2b double positive airway epithelial stem cells 7 days after injury, 11.42% versus 18.81% respectively. The FACs data also showed that the same population of airway epithelial stem cells was isolated by sorting for either the Scgb1a1-Sftpc markers or Scgb1a1-Fgfr2b markers.

### Example 7

This example illustrates that airway epithelial stem cells are positive for FgfR2b. Using a FACs assay, immunostaining results indicated that 3 days after injury of lung epithelial cells from exposure to naphthalene, regenerating Clara cells adjacent to NEBs are FgfR2b positive in control (non-injured) and Fgf10 overexpressing lungs but not in sFgfr2b or Dkk1 overexpressing lungs. FgfR2b expression in Dkk1 overexpressing lungs could be rescued by simultaneous overexpression of Fgf10.

### Example 8

This example illustrates that for the cleaved Notch intracellular domain (NotchICD) that Notch signaling is activated in regenerating Clara cells/lung epithelial stem cells 3 days after injury at both the BADJs and NEBs using an immunostaining assay. Notch activation was determined to be increased in lungs overexpressing Fgf10 and inhibited in lungs overexpressing sFgfr2b or Dkk1 but was rescued upon overexpression of both Dkk1 and Fgf10. In addition, an induction in Snail expression in the regenerating Clara cells adjacent to NEBs in control lungs 3 days after naphthalene injury was detected. This induction in Snail was dramatically increased in lungs overexpressing Fgf10, inhibited in lungs overexpressing sFgfr2b or Dkk1, and rescued upon overexpression of both Dkk1 and Fgf10.

### Example 9

This example illustrates that Clara^{V} undergo a transient EMT in response to Fgf10 to generate lung epithelial stem cells. smMHC-Cre mice, expressing the Cre recombinase under control of the smooth muscle myosin heavy chain promoter, were crossed with Rosa26R-eYFP or Rosa26R-LacZ reporter mice. During normal homeostasis, smMHC-Cre;Rosa26R mice only labeled airway and vascular smooth muscle cells in the lung. After naphthalene injury the first observance of labeled epithelial cells was around 3 days after naphthalene injury, indicating that Clara cells are undergoing a transient EMT to generate lung epithelial stem cells or adopt epithelial stem cell properties. Under ideal conditions, the labeled clusters would expand to eventually give rise to the majority of regenerated Clara cells. The percentage of labeled Clara cells was in direct correlation with the extent of initial injury, as a robust activation of the pathways above would be necessary to ultimately activate the smooth muscle myosin heavy chain promoter and achieve sufficient levels of Cre recombinase expression. Rosa26R labeled Clara cells could be detected near both the NEBs as well as the BADJs.

### Example 10

This example illustrates the role of Notch signaling in the generation of lung epithelial stem cells. Scgba1a1-rtTa;Tet-O-Cre;Rosa26-NotchICD mice were generated, allowing for inducible overexpression of the NotchICD in Clara Cells. Non-injured lungs from mice harboring only one Rosa26-NotchICD allele looked normal but showed robust Snail expression in all Clara cells. However, Clara cells from lungs isolated from mice homozygous for the Rosa26-NotchICD domain underwent a complete EMT and lungs showed marked airway fibrosis. Interestingly, Fgf10 overexpression by itself could induce Notch activation as well as Snail expression in non injured lungs. Finally, smMHC-Cre;Rosa26R-LacZ mice were crossed with mice overexpressing sFgfr2b or Dkk1. Inhibition of Fgf10 signaling resulted in a complete lack of labeled epithelial cells. These results demonstrate that Fgf10 signaling is necessary for the generation of airway epithelial stem cells through the process of EMT.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

The foregoing description of the present invention has been presented for purposes of illustration. The description is not intended to limit the invention to the form disclosed herein. Consequently, variations and modifications commensurate with the above teachings, and the skill or knowledge of the relevant art, are within the scope of the present invention. The embodiments described hereinabove are further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art. Each publication and reference cited herein is incorporated herein by reference in its entirety.

What is claimed is:

1. A method to treat a condition related to injury of lung epithelial cells in a subject, comprising administering a compound that binds fibroblast growth factor receptor 2b (FGfR2b).

2. A method to treat a condition related to injury of lung epithelial cells in a subject, comprising the steps of:
a. isolating lung epithelial stem cells (LESCs) expressing fibroblast growth factor receptor 2b (FGfR2b) from a donor;
b. culturing the LESCs expressing FgfR2b to multiply them; and
c. introducing the cultured LESCs expressing FgfR2b into the subject.

3. The method of claim 2, wherein during step (b) the LESCs are exposed to a compound that binds FGfR2b.

4. The method of claim 2, wherein LESCs are isolated by contacting with an antibody specific for FgfR2b.

5. The method of claim 2, wherein the donor is the subject.

6. The method of claim 1, wherein the compound is an agonist of FgfR2b.

7. The method of claim 6, wherein the compound is an antibody.

8. The method of claim 6, wherein the compound is a fibroblast growth factor (Fgf).

9. The method of claim 8, wherein the Fgf is selected from the group consisting of Fgf1, Fgf3, Fgf7, Fgf9, Fgf10, and Fgf22.

10. The method of claim 8, wherein the compound is Fgf10.

11. The method of claim 8, wherein the compound is a fragment of a Fgf capable of binding FgfR2b.

12. The method of claim 1, wherein the compound binding FgfR2b stimulates proliferation of LESCs expressing FgfR2b.

13. The method of claim 1, wherein the condition related to injury of lung epithelial cells in a subject is selected from the group consisting of asthma, inflammation of the lungs, a condition associated with exposure to environmental toxins, a condition associated with exposure to bacteria, a condition associated with exposure to a virus, cystic fibrosis, a pneumonectomy and bleomycin mediated epithelial injury.

14. The method of claim 13, wherein the condition related to injury of lung epithelial cells in a subject is a condition associated with exposure to environmental toxins and wherein the environmental toxin comprises a toxin selected from the group consisting of naphthalene, ozone, smoke, tobacco smoke, chemical fumes, exhaust, mustard gas, acid, aromatic hydrocarbons and radiation.

15. The method of claim 1, wherein the compound is administered by inhalation.

16. A method to detect lung injury in a subject, comprising detecting the level of expression of FgfR2b in a subject sample wherein an elevated level of expression of FgfR2b is indicative of lung injury.

17. The method of claim 16, wherein the lung injury is selected from group consisting of asthma, a condition associated with exposure to environmental toxins, a condition associated with exposure to bacteria, a condition associated with exposure to a virus, cystic fibrosis, a pneumonectomy and bleomycin mediated epithelial injury.

18. The method of claim 17, wherein the lung injury is a condition associated with exposure to environmental toxins and wherein the environmental toxin comprise s a toxin selected from the group consisting of naphthalene, ozone, smoke, tobacco smoke, chemical fumes, exhaust, mustard gas, acid, aromatic hydrocarbons and radiation.

19. A pharmaceutical composition comprising a compound that binds FgfR2b and a pharmaceutically acceptable carrier.

20. The pharmaceutical composition of claim 19, wherein the compound is an agonist of FgfR2b.

21. The pharmaceutical composition of claim 19, wherein the compound is a fibroblast growth factor (Fgf).

22. The pharmaceutical composition of claim 19, wherein the compound is selected from the group consisting of Fgf1, Fgf3, Fgf7, Fgf9, Fgf10, Fgf22 and a fragment of a Fgf capable of binding FgfR2b.

23. The pharmaceutical composition of claim 20, wherein the agonist of FgfR2b is an antibody.

24. A method to treat a condition related to proliferation of lung epithelial cells in a subject, comprising administering a compound that binds to FgfR2b, to block receptor signaling.

25. The method of claim 24, wherein the blocking of receptor signaling occurs by preventing dimerization of FgfR2b.

26. The method of claim 24, wherein the compound is an antibody.

27. The method of claim 24, wherein the compound comprises a cytotoxic agent.

28. The method of claim 1, wherein the subject is human.

29. The method of claim 3, wherein the compound is an agonist of FgfR2b.

30. The method of claim 3, wherein the compound binding FgfR2b stimulates proliferation of LESCs expressing FgfR2b.

31. The method of claim 2, wherein the condition related to injury of lung epithelial cells in a subject is selected from the group consisting of asthma, inflammation of the lungs, a condition associated with exposure to environmental toxins, a condition associated with exposure to bacteria, a condition associated with exposure to a virus, cystic fibrosis, a pneumonectomy and bleomycin mediated epithelial injury.
